(19) United States
(12) Patent Application Publication
(10) Pub. No.: US 2025/0239334 A1
(43) Pub. Date: Jul. 24, 2025

(54) GENERATIVE AI SYSTEMS FOR GENERATING CONTENT USING LARGE LANGUAGE MODELS

(71) Applicant: THE BOSTON CONSULTING GROUP, INC., Boston, MA (US)

(72) Inventors: Jakob Heyder, Boston, MA (US); Nigel Robert Markey, Boston, MA (US); Ilyass El Mansouri, Boston, MA (US); Casper Jacobus Johannes van Langen, Boston, MA (US); Christoph Meier, Boston, MA (US)

(21) Appl. No.: 18/420,188

(22) Filed: Jan. 23, 2024

(51) Int. Cl. G16H 10/20; G06N 3/0455

(52) U.S. Cl. CPC G16H 10/20; G06N 3/0455

(57) ABSTRACT

In an example method, a computer system accesses first data representing a plurality of first clinical trial protocols and second data representing a plurality of content generation tools. The system receives a user input instructing the computer system to generate a second clinical trial protocol using an LLM, where the user input includes an indication of a subject of the second clinical trial protocol. The system determines a plurality of actions to generate the second clinical trial protocol, and determines one or more content generation tools associated with each of the actions. The system causes the LLM to perform each of the actions using the one or more content generation tools associated with that action and based on the first data. Further, the system generates the second clinical trial protocol based on an output of the LLM, and stores a data structure representing the first second clinical trial protocol.

### BACKGROUND

In general, clinical trials are prospective biomedical or behavioral research studies that are designed to answer specific questions about biomedical or behavioral interventions. For example, clinical trials can be performed to evaluate the safety and/or efficacy of vaccines, drugs, dietary choices, dietary supplements, and/or medical devices.

### SUMMARY

In general, a computerized generative artificial intelligence (AI) system can be used to automatically generate documents for clinical trials using a large language model (LLM). For example, a system can automatically generate documents describing clinical trial protocols (e.g., procedures for performing clinical trials and/or interpreting the results of clinical trials). As another example, a system can automatically generate documents that are associated with clinical trials, such informed consent forms, clinical study reports, and/or any other documents that facilitate the performance of a clinical trial.

In an example implementation, a system provides several content generation tools that can be used in conjunction with a large language model (LLM) to generate content relating to clinical trials. As an example, these tools can include tools for searching for information from one or more data sources (e.g., the Internet, literature sources, drug databases, etc.). As another example, these tools can include tools for generating content for inclusion in one or more sections of a document (e.g., text content, graphical content, charts, graphs, tables, etc.). As another example, these tools can include tools for summarizing content generated by the system.

A user can instruct the system to generate one or more documents relating to a clinical trial, and provide information to guide the system in generating the documents. As an example, the user can specify the subject of the clinical trial (e.g., the intervention that is being tested), the types of clinical trial that is to be performed (e.g., the “phase” of the clinical trial), the intended audience of the document (e.g., a particular government agency), and/or any other information regarding the clinical trial.

Based on the user's instructions, the system selects one or more of the tools, and performs one or more actions using the selected tools to generate the requested documents. As an example, the system can select one or more tools to search for and retrieve relevant information regarding the subject of the clinical trial and example clinical trials that have been previously performed, and provide the information to the LLM. As another example, the system can select one or more tools to generate text, images, and/or other content based on the provided information, and instruct the LLM to generate the documents using those tools. As another example, the system can select one or more tools to generate a summary of the generated documents, instruct the LLM to generate a summary using those tools, and present the summary to the user (e.g., using a graphical user interface).

The implementations described herein can provide various technical benefits. As an example, the implementations described herein allow a computer system to automatically generate documents for clinical trials, without requiring that a user provide manual input regarding each and every portion of the document. As an example, based on a natural language prompt for a particular type of document, the system can automatically retrieve relevant information for the document, generate the document, and present a summary of the document without further manual input from a user. Accordingly, the computer system can generate data in a more efficient and/or effective manner (e.g., compared to computer systems that do not perform the techniques described herein).

Further, the implementations described herein allow a computer system to generate documents for clinical trials in a manner that would not be performed by a human, but is particularly suitable for performance by a computer. For example, a computer system can generate documents for clinical trials using a generative AI system having one or more LLMs, which is trained to identify statistical relationships from text documents during a computationally intensive self-supervised and semi-supervised training process. Based on the identified statistical relationships, the generative AI system can use the LLMs to generate content that is similar to that that would be produced by a human, without requiring that the computer system have the semantic or syntactic knowledge that a human might possess.

As another example, the implementations described herein include an improved graphical user interface that provides a user with information regarding the generation of documents using an LLM, in manner that is particularly intuitive for the user to understand. Accordingly, the operation of the computer system is improved (e.g., by increasing the case by which a user can interact with the computer system).

As another example, the implementation described herein can be used to improve the design and/or the performance of clinical trials (e.g., compared to those generated by systems that do not perform the techniques described herein). This allows researchers to better assess the effectiveness of medical interventions, thereby improving the safety and/or efficacy of the treatment of patients. For example, the techniques described herein can be used to automatically generate robust and comprehensive clinical trials protocols to accurately assess the safety and/or efficacy of a particular medial intervention. Based on this information, researchers can deploy the medical intervention to the public (e.g., if the medical intervention is deemed safe and effective), and/or modify the medical intervention to improve its safety and/or efficacy.

In an aspect, a method for generating clinical trial protocols using one or more computerized large language models (LLMs) includes: accessing, by a computer system from one or more hardware storage devices, first data representing a plurality of first clinical trial protocols; accessing, by the computer system from the one or more hardware storage devices, second data representing a plurality of content generation tools available for use with the one or more LLMs, where the one or more computerized LLMs include a generative transformer model having at least one of an encoder or a decoder; receiving, by the computer system, a user input instructing the computer system to generate a second clinical trial protocol using the one or more LLMs, where the user input comprises an indication of a subject of the second clinical trial protocol; determining, by the computer system, a plurality of actions to generate the second clinical trial protocol; determining, by the computer system, one or more content generation tools associated with each of the actions; causing, by the computer system, the one or more LLMs to perform each of the actions using the one or more content generation tools associated with that action and based on the first data, where performance of each of the actions causes the one or more LLMs to generate an output using at least one of the encoder or the decoder; generating, by the computer system, the second clinical trial protocol based on an output of the one or more LLMs; and storing, by the computer system using the one or more hardware storage devices, a data structure representing the second clinical trial protocol.

Implementations of this aspect can include one or more of the following features.

In some implementations, the method can further include presenting a graphical user interface representing the generation of the second clinical trial protocol.

In some implementations, presenting the graphical user interface can include continuously updating contents of the graphical user interface during the generation of the second clinical trial protocol.

In some implementations, the graphical user interface can include a plurality of first graphical display elements, each of the first graphical display elements representing a different respective one of the actions.

In some implementations, the graphical user interface can include a plurality of second graphical display elements, each of the second graphical display elements representing a summary of content generated for a different respective one of the actions.

In some implementations, the subject of the second clinical trial protocol can be one of a drug or a medical procedure.

In some implementations, the first data can further represent at least one of: a plurality of drugs, or a plurality of medical treatments.

In some implementations, the first data can include structured data, and accessing the first data can include at least one of: querying the structured data, normalizing the structured data, augmenting the structured data using the one or more LLMs, adding metadata to the structured data, or ingesting the structured data into a vector database.

In some implementations, the first data can include one or more documents, and accessing the first data can include at least one of: parsing the one or more documents, augmenting the one or more documents, segmenting the one or more documents into one or more portions, adding metadata to the one or more documents using the one or more LLMs, or ingesting the documents into a vector database.

In some implementations, the actions can include at least one of: searching the first data for content relevant to second clinical trial protocol, generating a preview of the second clinical trial protocol, or generating the second clinical trial protocol.

In some implementations, the content generation tools can include at least one of: a tool to perform a search of the first data, a tool to perform a search of one or more external databases, or a tool to perform a search of one or more websites.

In some implementations, the content generation tools can include at least one of: a tool to generate a title of the second clinical trial protocol, or a tool to generate one or more sections of the second clinical trial protocol.

In some implementations, the content generation tools can include a tool to generate a summary of the second clinical trial protocol.

In some implementations, the content generation tools can include a tool to generate at least one of a graph or a chart for the second clinical trial protocol.

In some implementations, the content generation tools can include a tool to prompt a user for additional user input regarding the second clinical trial protocol.

In some implementations, at least one of the encoder or the decoder can be configured to apply a computerized attention mechanism over its respective inputs while generating the second clinical trial protocol.

In some implementations, the method can further include: receiving a second user input instructing the computer system to generate one or more clinical trial documents using the one or more LLMs; determining a plurality of second actions to generate the one or more clinical trial documents; determining one or more second content generation tool associated with each of the second actions; causing the one or more LLMs to perform each of the second actions using the one or more second content generation tools associated with that second action, where performance of each of the second actions causes the one or more LLMs to generate a second output; generating the one or more clinical trial documents based on the second output of the one or more LLMs; and storing, using the one or more hardware storage devices, a second data structure representing the one or more clinical trial documents.

In some implementations, the one or more clinical trial documents can include a clinical study report.

In some implementations, the one or more clinical trial documents can include an informed consent form.

In another aspect, a method for generating clinical study reports using one or more computerized large language models (LLMs) includes: accessing, by a computer system from one or more hardware storage devices, first data representing a plurality of first clinical study reports; accessing, by the computer system from the one or more hardware storage devices, second data representing a plurality of content generation tools available for use with the one or more LLMs, where the one or more computerized LLMs include a generative transformer model having at least one of an encoder or a decoder; receiving, by the computer system, a user input instructing the computer system to generate a second clinical study reports using the one or more LLMs, where the user input include an indication of a subject of the second clinical study reports; determining, by the computer system, a plurality of actions to generate the second clinical study reports; determining, by the computer system, one or more content generation tools associated with each of the actions; causing, by the computer system, the one or more LLMs to perform each of the actions using the one or more content generation tools associated with that action and based on the first data, where performance of each of the actions causes the one or more LLMs to generate an output using at least one of the encoder or the decoder; generating, by the computer system, the second clinical study reports based on an output of the one or more LLMs; and storing, by the computer system using the one or more hardware storage devices, a data structure representing the second clinical study reports.

Other embodiments of this aspect include corresponding computer systems, apparatus, and computer programs recorded on one or more computer storage devices. A system of one or more computers can be configured to perform particular actions by virtue of having software, firmware, hardware, or a combination of them installed on the system that in operation causes or cause the system to perform the actions. One or more computer programs can be configured to perform particular actions by virtue of including instructions that, when executed by a data processing apparatus, cause the apparatus to perform the actions.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example system, including a clinical trial document generation engine, for automatically generating documents for clinical trials.

FIG. 2 shows an example clinical trial document generation engine.

FIG. 3 shows an example architecture of a clinical trial document generation engine.

FIG. 4 shows an example process for generating clinical trial documents.

FIG. 5 shows an example process for searching and collecting data for facilitating the generation of clinical trial documents.

FIGS. 6A-6D show example graphical user interfaces.

FIG. 7 illustrates an example process for generating clinical trial documents using a generative AI system.

FIG. 8 depicts an example computing system, according to implementations of the present disclosure.

Like reference numbers and designations in the various drawings indicate like elements.

### DETAILED DESCRIPTION

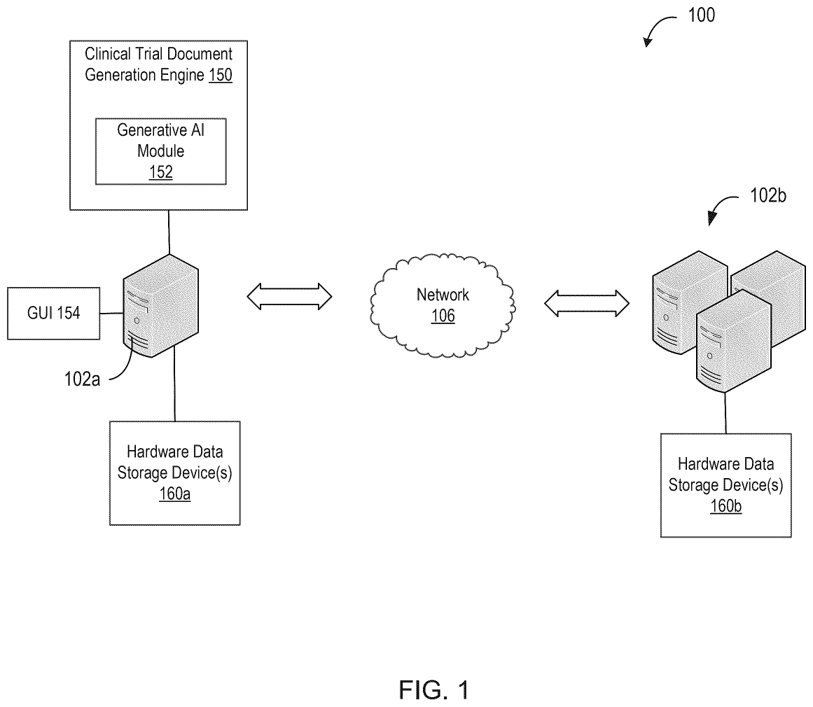

FIG. 1 shows an example system 100 for automatically generating documents for clinical trials using a large language model (LLM). For example, the system 100 can automatically generate documents describing clinical trial protocols (e.g., procedures for performing clinical trials and/or interpreting the results of clinical trials). As another example, the system 100 can automatically generate documents that are associated with clinical trials, such informed consent forms, clinical study reports, and/or any other documents that facilitate the performance of a clinical trial.

The system includes a clinical trial document generation engine 150 implemented on a computer system 102a. The engine 150 includes a generative artificial intelligence (AI) module 152 having one or more LLMs. Example LLMs include models having or more generative pre-trained transformers (GPTs), such as those implemented using or more artificial neural networks.

In general, the engine 150 provides several content generation tools that can be used in conjunction with the generative AI module 152 to generate content relating to clinical trials. As an example, these tools can include tools for searching for information from one or more data sources (e.g., the Internet, literature sources, drug databases, etc.). As another example, these tools can include tools for generating content for inclusion in one or more sections of a document (e.g., text content, graphical content, charts, graphs, tables, etc.). As another example, these tools can include tools for summarizing content generated by the system.

During an example operation of the system 100, a user instructs the engine 150 to generate one or more documents relating to a clinical trial, and provides information to guide the system in generating the documents. As an example, using a graphical user interface (GUI) 154, the user can specify the subject of the clinical trial (e.g., the intervention that is being tested), the types of clinical trial that is to be performed (e.g., the “phase” of the clinical trial), the intended audience of the document (e.g., a particular government agency), and/or any other information regarding the clinical trial. In some implementations, the user can input natural language commands (e.g., in the form of sentences and/or phrases) using the GUI 154, such as by inputting text into a text box of the GUI 154. In some implementations, the user can provide documents, articles, literature, or other content to the engine 150 (e.g., using the GUI 154, such as by uploading content using the GUI 154).

Based on the user's instructions, the engine 150 selects one or more tools, and performs one or more actions using the selected tools and the generative AI module 152 to generate the requested documents.

For instance, the engine 150 can select one or more tools to search for and retrieve relevant information regarding the subject of the clinical trial and example clinical trials that have been previously performed, and provide the information to the generative AI module 152. As an example, the tools can be used to search for and retrieve data from one or more hardware data storage device(s) 160a local to the computer system 102a (e.g., content uploaded by the user, locally stored content retrieved from one or more other sources, etc.). As another example, the tools can be used to search for and retrieve data from one or more hardware data storage device(s) 160b implemented on one or more computer systems 102b remote from the computer system 102a (e.g., remote servers, cloud computing platforms, etc.). For example, the engine 150 can use a network 106 to communicate with the computer systems 102b (e.g., in order to issue commands or requests to the computer systems 102b, receive data from the computer systems 102b, etc.).

Further, the engine 150 can select one or more tools to generate text and/or images based on the provided information, and instruct the generative AI module 152 to generate the documents using those tools. For example, the engine 150 can instruct the generative AI module 152 to generate one or more portions of text, images, graphs, charts, tables, or another other content, can arrange the generated content in one or more documents.

Further, the engine 150 can select one or more tools to generate a summary of the generated documents, instruct the generative AI module 152 to generate a summary using those tools, and present the summary to the user (e.g., using the GUI 154). In some implementations, the engine 150 can use the generative AI module 152 to generate and update the summaries during the generation of the documents (e.g., in real time) in order to provide the user with up to date information regarding the progress of the document generation process.

In some implementations, at least some of the generated documents can be stored locally (e.g., using the hardware data storage devices(s) 160a local to the computer system 102a). In some implementations, at least some of the generated documents can be stored remotely (e.g., using the computer systems 102b and hardware data storage devices 160b remote from the computer system 102a).

In general, each of the computer systems 102s and 102b can include any number of electronic devices that are configured to receive, process, and transmit data. Examples of the computer systems include client computing devices (e.g., desktop computers or notebook computers), server computing devices (e.g., server computers or cloud computing systems), mobile computing devices (e.g., cellular phones, smartphones, tablets, personal data assistants, notebook computers with networking capability), wearable computing devices (e.g., smart phones or headsets), and other computing devices capable of receiving, processing, and transmitting data. In some implementations, the computer systems can include computing devices that operate using one or more operating systems (e.g., Microsoft Windows, Apple macOS, Linux, Unix, Google Android, and Apple IOS, among others) and one or more architectures (e.g., x86, PowerPC, and ARM, among others). In some implementations, one or more of the computer systems need not be located locally with respect to the rest of the system 100, and one or more of the computer systems can be located in one or more remote physical locations.

Each the computer systems 102a and 102b can include a respective user interface (e.g., GUI 154) that enables users interact with the computer system, other computer systems, and/or the engine 150. Example interactions include viewing data, transmit data from one computer system to another, and/or issuing commands to a computer system. Commands can include, for example, any user instruction to one or more of the computer system to perform particular operations or tasks. In some implementations, a user can install a software application onto one or more of the computer systems to facilitate performance of these tasks.

In FIG. 1, the computer system 102a is illustrated as a single component. However, in practice, the computer system 102a can be implemented on one or more computing devices (e.g., each computing device including at least one processor such as a microprocessor or microcontroller). As an example, the computer system 102a can be a single computing device that is connected to the network 106, and the engine 150 can be maintained and operated on the single computing device. As another example, the computer system 102a can include multiple computing devices that are connected to the network 106, and the engine 150 can be maintained and operated on some or all of the computing devices. For instance, the computer system 102a can include several computing devices, and the engine 150 can be distributed on one or more of these computing devices.

The network 106 can be any communications network through which data can be transferred and shared. For example, the network 106 can be a local area network (LAN) or a wide-area network (WAN), such as the Internet. The network 106 can be implemented using various networking interfaces, for instance wireless networking interfaces (such as Wi-Fi, Bluetooth, or infrared) or wired networking interfaces (such as Ethernet or serial connection). The network 106 also can include combinations of more than one network, and can be implemented using one or more networking interfaces.

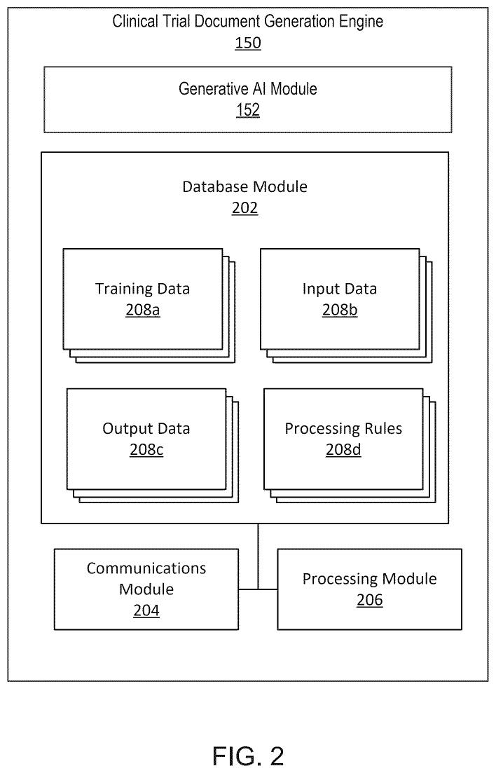

FIG. 2 shows various aspects of the clinical trial document generation engine 150. In general, the engine 150 includes several operation modules that perform particular functions related to the operation of the engine 150. For example, the engine 150 includes a generative AI module 152. Further, the engine 150 includes a database module 202, a communications module 204, and a processing module 206. The operation modules can be provided as one or more computer executable software modules, hardware modules, or a combination thereof. For example, one or more of the operation modules can be implemented as blocks of software code with instructions that cause one or more processors of the engine 150 to execute operations described herein. In addition or alternatively, one or more of the operations modules can be implemented in electronic circuitry such as, e.g., programmable logic circuits, field programmable logic arrays (FPGA), or application specific integrated circuits (ASIC).

The database module 202 maintains information related to generating clinical trial documents using the generative AI module 152.

As an example, the database module 202 can store training data 208a for training or prompting the generative AI module 152. In some implementations, the training data 208a can include example clinical trial documents, such as those previously generated by the engine 150 and/or those manually produced by one or more human users. For instance, the training data 208a can include documents describing clinical trial protocols, informed consent forms, clinical study reports, and/or any other documents that facilitates the performance of a clinical trial.

As another example, the database module 202 can store input data 208b that is used as an input to the generative AI module 152. As an example, the input data 208b can include commands or instructions provided by a user, including information regarding a particular desired output of the engine 150. For instance, the input data 208b can include information regarding the subject of the clinical trial (e.g., the intervention that is being tested), the types of clinical trial that is to be performed (e.g., the “phase” of the clinical trial), the intended audience of the document (e.g., a particular government agency), and/or any other information regarding the clinical trial.

Further, the input data 208b can include information retrieved by the engine 150 in support of the document generation process. As an example, the input data 208b can include data retrieved from one or more drug information databases (e.g., information regarding a drug's composition, interactions between drugs, dosage of drugs, indications for use, side effects, etc.).

As another example, the input data 208b can include data retrieved from one or more from one or more medical or scientific journals. For instance, the input data 208b can one or more articles or other publications describing the use, safety, and/or efficacy of certain drugs or other medical interventions.

As another example, the input data 208b can include data regarding one or more existing clinical trial protocols. For instance, the input data 208b can include data regarding series of steps, procedures, actions, or operations that were previously performed (e.g., by clinical trial researchers) to assess the safety and/or efficacy of particular medical interventions.

As another example, the input data 208b can include data regarding one or more rules, regulations, and/or guidelines for performing clinical trials. For instance, the input data 208b can include data regarding government rules, regulations and/or guidelines (e.g., as specified by a government agency, such as the Federal Drug Administration, FDA). The input data 208b can also include data regarding institutional rules, regulations and/or guidelines (e.g., as specified by a public or private hospital).

Further, the database module 202 can store output data 208c generated by the generative AI module 152. As an example, the output data 208c can include one or more portions of content (e.g., text, images, charts, graphs, tables, etc.) generated by the generative AI module 152 based on the input data 208b. As another example, the output data 208c can include one or more document generated by the generative AI module 152 based on the input data 208b.

Further, the database module 202 can store processing rules 208d specifying how data in the database module 202 can be processed to generate clinical trial documents using the generative AI module 152.

As an example, the processing rules 208d can include one or more rules for implementing, instruction tuning or prompting, and operating the generative AI module 152 to produce the output data 208c. For example, the one or more rules can specify that the training data 208a be provided to the generative AI module 152 for training or prompting (e.g., such that the generative AI module 152 can identify trends and/or correlations between the contents of clinical trial documents and the subject matter therein, and generate new output based on those identified trends and/or correlations).

As another example, the one or more rules can specify that the input data 208b be provided to the generative AI module 152 (e.g., to generate output data 208c representing the contents of a clinical trial document).

As another example, the one or more rules can specify that the generated output data 208c be presented to the user and/or stored for future retrieval and/or processing (e.g., using the database module 202).

As another example, the one or more rules can specify one or more tools that facilitate the performance of particular actions by the generative AI module 152. For example, the tools can specify certain actions or operations that can be performed by the generative AI module 152 to retrieve data and to generate content based on the retrieved data.

Example data processing techniques are described in further detail below.

As described above, the engine 150 also includes a communications module 204. The communications module 204 allows for the transmission of data to and from the engine 150. For example, the communications module 204 can be communicatively connected to the network 106, such that it can transmit data to and receive data from the computer system 102b. Information received from the computer system 102b can be processed (e.g., using the processing module 206) and stored (e.g., using the database module 202).

As described above, the engine 150 also includes a processing module 206. The processing module 206 processes data stored or otherwise accessible to the engine 150. For instance, the processing module 206 can be used to execute one or more of the operations described herein (e.g., operations associated with the generative AI module 152).

In some implementations, a software application can be used to facilitate performance of the tasks described herein. As an example, an application can be installed on the computer systems 102a. Further, a user can interact with the application to input data and/or commands to the engine 150, and review data generated by the engine 150.

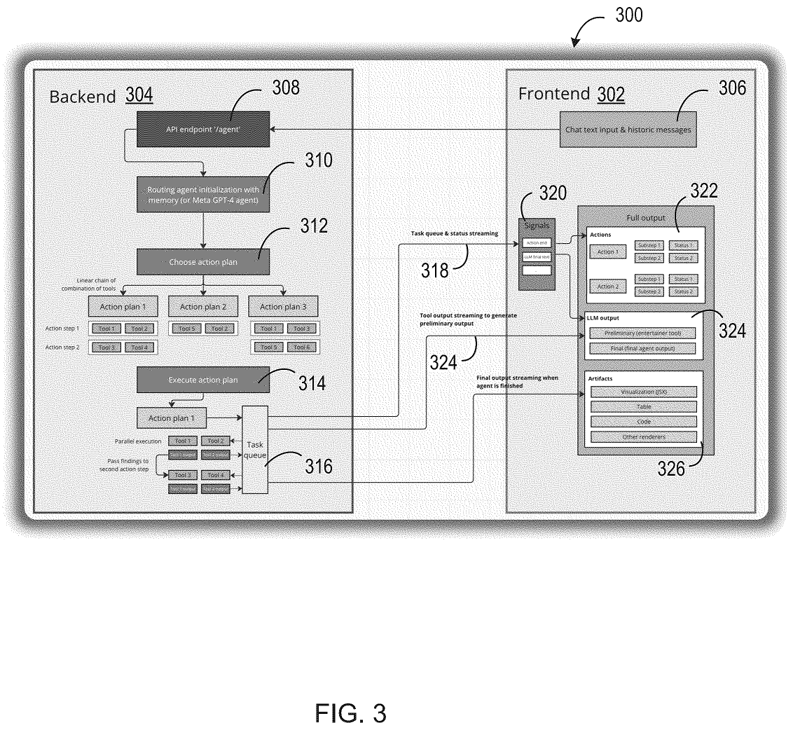

FIG. 3 shows an example architecture 300 of a clinical trial document generation engine 150. In general, the architecture 500 includes a frontend 302 for interacting with a user, and a backend 304 for processing data (e.g., to process data input by the user and to process data for presentation to the user).

According to the architecture 300, a user provides chat text input and/or historic messages to the engine 150 (306). As an example, the user can enter natural language text (e.g., sentences and/or phrases) specifying the type of document that should be generated by the engine 150, the subject of the document (e.g., the subject of a particular clinical trial, such as the particular medical intervention that is being tested), the type of clinical trial that is to be performed (e.g., the “phase” of the clinical trial), the intended audience of the document (e.g., a particular government agency), and/or any other information regarding the clinical trial. In some implementations. As another example, the user can enter any pertinent background information relating to the clinical trial and/or the medical intervention.

The backend 304 receives the user's input via an application programming interface (API) (308). In some implementations, the API may also be referred to as an “agent.” In general, the API specifies the manner in which data can be input into and/or output from the backend 304 (e.g., by providing particular software functions and/or platforms for interacting with the backend 304). In some implementations, the backend 304 can parse the user's input, and convert the user's input into a format or data structure that can be further interpreted and/or processed by the backend 304.

Further, a routing agent is initialized by the backend 304 (310). The routing agent stores the user's input (e.g., in memory). Further, the routing agent selects one or more actions to be performed based on the user's input (312).

In general, multiple actions can be combined together to perform a particular task (also referred to as an “action plan”). For example, each combination of actions can include processing data using one or more specific tools. In some implementations, each combination of actions can include using certain tools in a particular order or sequence. For example, a first tool can be used to generate a first set of data, which in turn can be provided to a second tool to generate a second set of data, and so forth.

In some implementations, the routing agent can retrieve or generate multiple action plans, each having a different combination of tools and/or actions. Further, the routing agent can select and execute one of more of the action plans (314).

For instance, in the example shown in FIG. 3, the routing agent can select a first action plan (“Action plan 1”) having several actions and corresponding tools (“Tool 1,” “Tool 2,” “Tool 3,” and “Tool 4”). Further, the routing agent can queue the first action plan (and its constituent actions) into a task queue 316 for execution. For example, actions can be performed using a first set of tools (e.g., “Tool 1” and “Tool 2”) to generate a first set of output (e.g., “Tool 1 output” and “Tool 2 output”). Further, the first set of put can be provided to a set of tools (e.g., “Tool 3” and “Tool 4”) to generate a second set of output (e.g., “Tool 3 output” and “Tool 4 output”). The output of one or more of the actions and tools can be provided to the task queue 316 for further processing and/or for presentation to the user.

As an example, at least some of the data stored in the task queue 316 can be provided to the frontend 302 to present information to the user regarding the status of the document generation process (318). For instance, at least some of the data stored in the task queue 316 can be used to generate signals 320 indicating the actions and/or tools that were selected for execution (and any sub-steps thereof), and the status of the execution of each of those actions and/or tools. These signals 320 can be used to generate output for presentation to the user (e.g., via the GUI 154) (322). For example, the GUI 154 can include graphical elements that visually indicate the actions and/or tools that were selected for execution (and any sub-steps thereof), and the status of the execution of each of those actions and/or tools. Further, the GUI 154 can be continuously, periodically, or intermittently updated to reflect any changes to the status of the document generation process.

As another example, at least some of the data stored in the task queue 316 can be provided to the frontend 302 to present preliminary information to the user regarding the content that is being generated by the engine 150 (324). For instance, as content is being generated by the engine 150 (e.g., via execution of the selection action(s) and/or tool(s)), at least some of the generated content can be provided to the frontend 302 for presentation to the user (e.g., via an “entertainer tool” or agent). This can be useful, for example, in providing the user with real time (or substantially real time) feedback regarding the progress of the content generation process.

Further, upon the completion of the content generation process by the engine 150, at least some of the generated content can be provided to the frontend 302 for presentation to the user (e.g., “final agent output”). This can be useful, for example, in providing the user with feedback regarding the final version of the generated content.

In some implementations, the GUI 154 can include graphical elements that visually indicate the generated content (e.g., as the content is being generated and/or upon the completion of the content generation process). For example, the GUI 154 can display text, images, charts, graphs, tables, and/or any other content, such as in the form of an electronic document.

As another example, at least some of the data stored in the task queue 316 can be provided to the frontend 302 to present information regarding any additional content (e.g., “artifacts”) that has been generated by the engine 150 (326). For example, at least some of the data can be used to generate visualizations (e.g., data visualizations, such as that rendered using JavaScript and/or JSX), tables, code (e.g., computer code), and/or any other content.

Further, at least some of the generated content is also stored for future retrieval and/or processing (e.g., using one or more hardware data storage devices 160a and/or 160b).

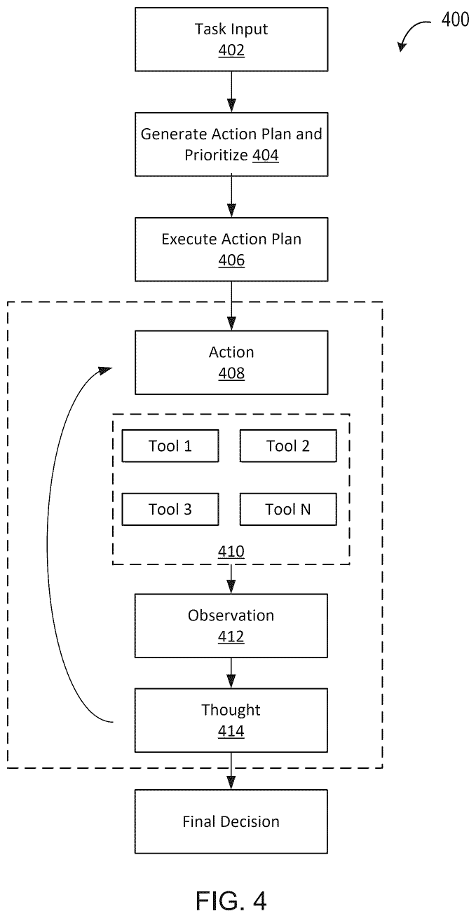

An example process 400 for generating clinical trial documents using the engine 150 is shown in FIG. 4. The process 400 can be performed, at least in part, using one or more agents of the architecture 300 (e.g., using the backend 304).

In general, agents (e.g., software agents) can be configured to solve complex tasks by planning and executing a set of actions using artificial intelligence. As an example LLM-based agents can be configured to leverage the ability of LLMs to decide on actions based on the tools they are given. Further, agents can solve more complex problems than an independent LLM request. Further, agents can also overcome known LLM limitations, such as access to recent information, math problems, etc., due in part to their access to specialized tools (e.g., web search, calculators, etc.).

At an example, the architecture 300 can include one or more agents that have access to a suite of tools and LLMs, and that are configured to determine which tool to use based on a user's input.

As another example, the architecture 300 can include one or more agents that are configured to determine the action(s) to take and execute those action(s) (e.g., one at a time in a particular sequence). These agents may be referred as “action agents.”

As another example, the architecture 300 can include one or more agents that are configured to determine a plan of actions to take, and then execute those action(s) (e.g., one at a time in a particular sequence). These agents may be referred as a “plan and execute agents.”

In the process 400, a system receives task input from a user (402). As an example, the task input can include one or more questions or prompts from the user (e.g., a question from the user request further information regarding a particular topic, a prompt to generate a document or content regarding a particular subject, etc.).

The system generates an action plan based on the user's input (404) and executes the action plan (406). In general, as described with reference to FIG. 3, an action plan can include one more actions (or sub-actions). Further, each action can be associated with one or more tools. For example, referring to FIG. 4, an action 408 can be associated with one or more tools 410.

In some implementation, the system can generate an action plan based, at least in part, on the actions and/or tools that are available to the agent. Further, in at least some implementation, the system can generate an action plan based, at least in part, on feedback from one or more LLMs. For example, the system can provide an LLM with (i) a set of available actions and/or tools can be performed, and (ii) request that the LLM select one or more actions and/or tools to achieve a particular result or output (e.g., searching and for and/or retrieving particular data, generating particular content for inclusion in a document, etc.). Based on the provided information, the LLM can select one or more of the actions and/or tools for execution by the system. In some implementations, the LLM can also determine a particular order or sequence in which the selected actions and/or tools can be executed to achieve the specified result or output.

Further, the system extracts the most relevant point(s) from the question context that will define the answer and/or produces the result of the action (412). This may be referred as an “observation” step.

Further, the system determines what to do and/or shares a long answer derivation logic (414). This may be referred as an a “thought” step.

In some implementations, the system can repeat performing actions (e.g., action(s) 408), observations 412, and/or thought 414 until a final decision is determined (416). In some implementations, the final decision can include one or more clinical trial documents (or one or more portions thereof). Further, at least some of the documents and/or portions of the documents can be presented to the user for review (e.g., using the GUI 154). Further still, at least some of the documents and/or portions of the documents can be stored for future retrieval and/or processing (e.g., using one or more hardware data storage devices 160a and/or 160b).

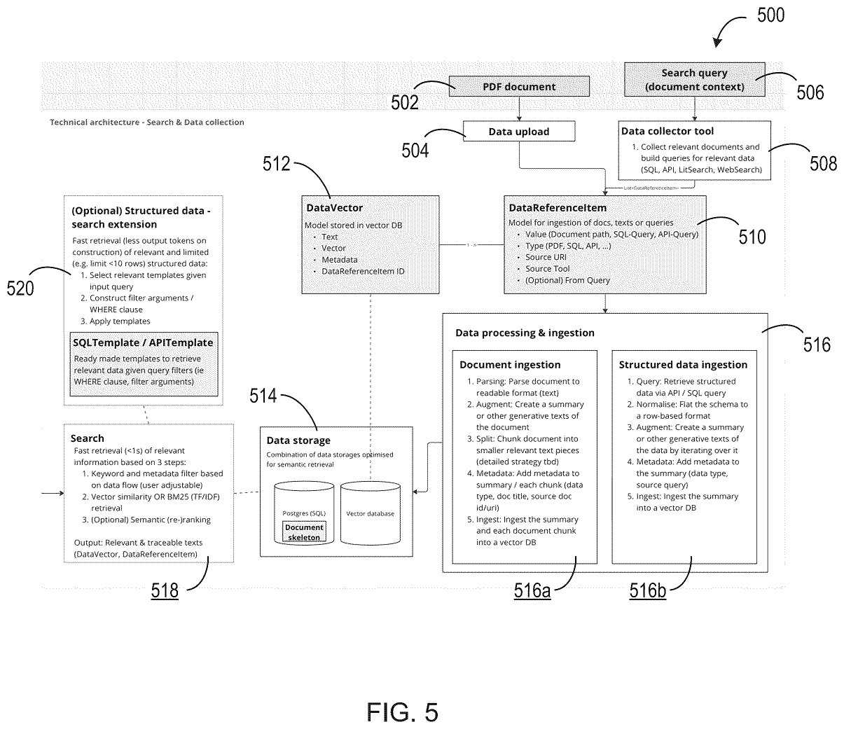

An example architecture 500 for search and collecting data using the engine 150 is shown in FIG. 5.

In general, the engine 150 can collect data from one or more electronic documents or files 502, such as a Portable Document Format (PDF) documents. For example, one or more documents or files can be uploaded to the engine 150 for processing (504). In some implementations, at least some of the documents can be manually uploaded ot the engine 150 by a user.

Further, the engine 150 can also search for data from one or more documents or files. For example, the engine 150 can generate a search query (506) and provide the search query to a data collector tool (508). Based on this information, the data collector tool can collect relevant documents (e.g., documents and/or files uploaded to the engine 150) and/or build queries for relevant data. In some implementations, documents can be collected and searched for using Structured Query Language (SQL) and/or using an API. In some implementations, searches can be performed with respect to collections of scientific, medical, or academic literature. In some implementations, searches can be performed with respect to documents obtained from the Internet or WQrld Wide Web (WWW).

The engine 150 generates one or more data structures based on the collected items (e.g., documents and/or files collected through uploading and/or via a search) (510). For example, the data structure can arrange information regarding each of the items in a format or model that facilitates ingestion by the engine 150. For example, for each item, the data structure can indicate a value (e.g., a document path, SQL-Query, API-Query, etc.), a data type (e.g., PDF, SQL, API, etc.), a source Uniform Resource Identifier (URI), and/or a source tool. Further, in at least some implementations, the data structure can indicate a “From Query” representing a history of queries used to reach the current action item. In some implementations, this data structure may be referred to as a “DataReferenceItem” data structure.

Further, the system generates one or more data vectors based on the data structures (512). For example, for each of the items, at least some of the item's contents (e.g., text, images, etc.) can be stored as one or more elements of the data vector. Further, at least some of the information from the item's data structure can be stored in one or more additional elements of the data vector. In some implementations, for each item (e.g., document or file), a data vector can include the contents of the item (or a portion thereof), metadata regarding the tile, and the item's data structure (e.g., the item's “DataReferenceItem” data structure).

At least some of the data vectors can be stored in a vector database for future retrieval and processing (514). For example, one or more data vectors can be stored using a relational database management system (e.g., SQL, Postgres, etc.). As another example, one or more data vectors can be stored using a vector database.

Further, the system also processes and ingests the items based on the data structures (516).

For example, the system ingests at least some of the documents (e.g., PDF documents) (516a). This ingestion process includes:
(i) Parsing: parsing the documents into a readable format (e.g., text, such as plaintext),
(ii) Augmenting: creating a summary or other generative texts of the document,
(iii) Splitting: chucking or segmenting the document into smaller relevant text pieces),
(iv) Generating metadata: adding meta to the summary of the document and/or to each chuck of the document (e.g., metadata indicating the data type, document tile, source document ID or URL, etc.), and
(v) Ingesting: ingesting the summary and each document chunk into a vector database.

At least some of the ingested information can be stored in a vector database for future retrieval and processing (514).

Further, the system also ingests at least some of the structured data obtained during the data collection process (516b). This ingestion process includes:
(i) Querying: retrieving structure data (e.g., via API and/or SQL queries),
(ii) Normalizing: flatting the schema of the data to a row-based format,
(iii) Augmenting: creating a summary or other generative texts of the data by iterating over it (e.g., using an LLM),
(iv) Generating metadata: adding metadata to the summary (e.g., metadata indicating a data type, source query, etc.), and
(v) Ingesting: ingesting the summary into a vector database.

At least some of the ingested information can be stored in a vector database for future retrieval and processing (514).

In general, the engine 150 can also obtain data via a “fast retrieval” search of data, such as structured or unstructured data in a database (518). As an example, a fast retrieval search can be performed based by:
(i) Filtering data using a keywords and/or metadata filter based on data flow,
(ii) Determining vector similar and/or Best Matching 25 (BM25) with respect to Term Frequency-Inverse Document Frequency (TF-IDF), and
(iii) Optionally, performing semantic ranking or re-ranking.

At least some of the obtained data can be stored in a vector database for future retrieval and processing (514).

In some implementations, a search can be ep formed using a structured data-search extension (520). For example, a fast retrieval search can be performed to identify relevant information in structured data by:
(i) Selecting relevant templates given an input query,
(ii) Constructing filter arguments/WHERE clauses, and
(iii) Applying the templates.

In some implementations, pre-generated templates can be used to retrieve relevant data given queried filter (e.g., templates using a particular WHERE clause, filter arguments, etc.). In some implementations, the templates can be used to search for data from an SQL data and/or using one or more APIs.

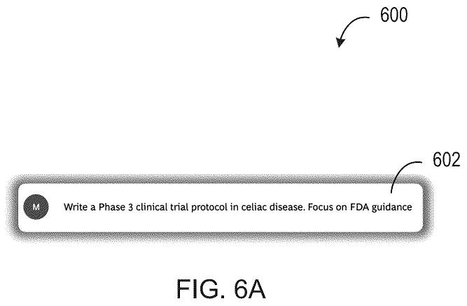

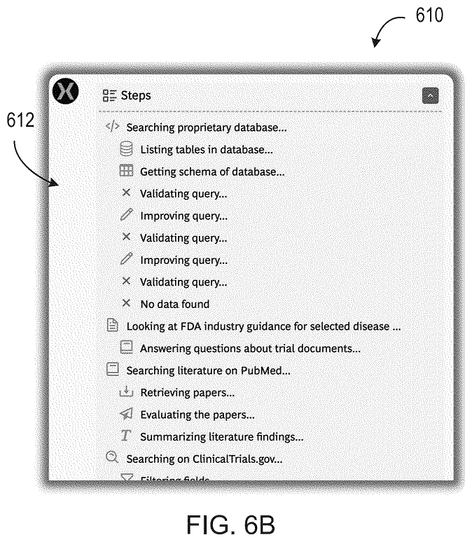

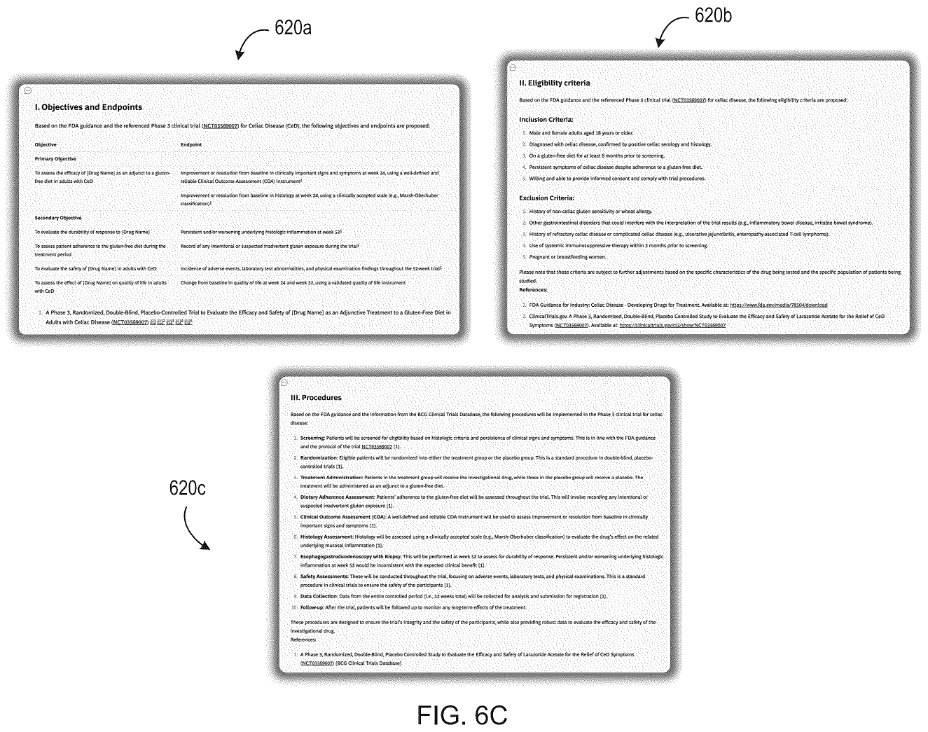

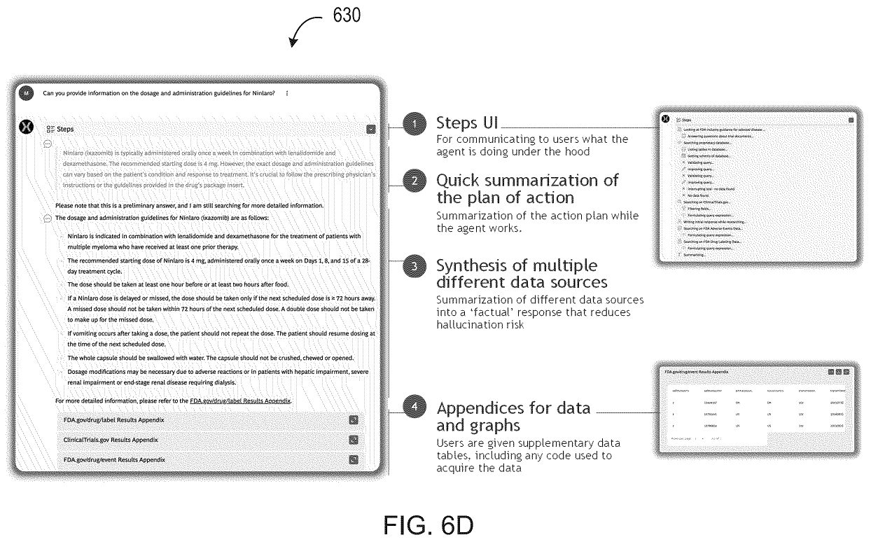

As described above, the engine 150 can generate a GUI to receive input from a user and/or present output to a user as a part of a clinical trial document generation process. Example GUIs are shown in FIGS. 6A-6D.

FIG. 6A shows an example GUI 600 for reciting input from a user. For example, the GUI 600 can include an input box 602 for receiving text input from a user (e.g., in the from of a chat message or other textual input). In this example, the user has entered a chat message with a request to generate a Phase 3 clinical trial protocol in celiac disease, with a focus on FDA guidance.

FIG. 6B shows an example GUI 610 for presenting status information to the user. For example, the GUI 610 includes several display elements 612, each indicating a respective action or step of a document generation process. Further, each of the display element 612 can indicate a status of its action or step (e.g., whether the action or step is in progress, has been complete, has encountered in error, etc.). In general, the system can update the GUI 610 during the document generation process (e.g., continuously, periodically, intermittently, etc.) to reflect any changes to the status of each of the actions or steps.

FIG. 6C shows an example GUIs 620a-620c for presenting output to a user. For example, the GUIs 620a-620c can display text, images, or other content, such as in the form of an electronic document.

In some implementations, the engine 150 can generate a GUI that presents preliminary information to a user, while the document generation process is still underway. This can be useful, for example, in providing the user with real time (or substantially real time) feedback throughout the content generation process. As an example, as shown in FIG. 6D, a GUI 630 in indicate each of the steps that are have already been performed, are currently being performed, or will be performed by the engine 150. Further, the GUI 630 can provide a summary of the plan of action that is being performed by the engine 150 (e.g., a natural language summary). Further, the GUI 630 can provide a summary of multiple different data sources, such as by synthesizing the data sources into a factual response that reduces “hallucination” risk on the part of the LLM (e.g., a risk that the LLM generates incorrect information). Further, the GUI can present appendices for data and graphs (e.g., supplementary data tables, including any code used the acquire the data).

In general, the system can update the GUI 640 during the document generation process (e.g., continuously, periodically, intermittently, etc.) to reflect any changes to the status of each of the actions or steps.

### Example Tools

In general, tools can be used by the engine 150 to search for data, retrieve data, generate content, and/or output content (e.g., in order to generate one or more clinical trial documents). In some implementations, a tool can include certain inputs, prompts, or commands to a LLM to provide a particular output. In some implementations, at least some of the inputs, prompts, or commands can include natural language text (e.g., natural language text that instructs the LLM to generate particular output, given particular input information, conditions, assumptions, etc.).

Several example tools are described in further detail below.
combined search tool: This is a research tool that is useful for searching data sources for scientific information regarding clinical trials. Based on an input search query, this tool can be used to search in multiple sources for relevant information (e.g., web-search, literature-search, clinical trials database, clinicaltrials.gov API, etc.). The tool outputs the relevant information found including the source. This tool can include one or more of the search tools described below, such as “web_search_tool,” “sql_tool,” “api_tool,” “pdf_tool,” “literature_search_tool.”
web search tool: This tool receives an input search query, and returns snippets from web search.
literature search tool: This tool receives a question (e.g., a natural language question), and returns an answer from a literature search.
protocol writing tool: This is an “expert” tool that is used to answer general questions given information as context. The input for the tool is a user question and context information as a long string, which should state some design parameters of a clinical trial. The output of the tool is an answer to the user question in the form of text grouped into sections of a clinical trial protocol.
protocol section writing tool: This tool is used to draft a part of a specific protocol section. The input to this tool is a string, which includes instruction to the tool to write a particular section. The tool can have access to the latest research information returned by “combined_search_tool”. Further, the researched information can be referenced in the instructions. Additional information can be provided to the tool as input to guide the drafting process. The output of the tool is a draft of the section part requested in the objective.
expert tool: This is an “expert” tool that is used to answer general questions given information as context. The input for the tool is a user question and context information as a long string. The output of the tool is an answer to the user question.
entertainer tool: This tool is used to generate a small concise output from memory while the information is loading. The input to the tool is a user question. The output of the tool is an answer to the user question.
summarizer tool: This tool is used to reduce the length of a portion of text to a few sentences. The input to the tool is a long string. The output of the tool is a short summary of the input.
visualizer tool: This tool is used to display data as a graph or chart using JSX code. The tool can visualize the result of SQL queries or API calls by providing recharts-JSX code. The input to the tool is a user query and information needed to visualize (e.g., SQL or API results). The output of the tool is a string that contains JSX code (e.g., to generate the graph or chart) in markdown format.
sql tool: This tool is used to query a clinical trial protocol database (e.g., an extended version of the ClinicalTrials.gov database). The database can include details on study design, eligibility criteria, interventions, study locations, and contact information for study investigators. I can also provide information on the trial's status (e.g., recruiting, active, completed), results, and links to relevant publications. Additional, the database can contain extra information about the complexity of a trial. The input of the tool is a question about the data in natural language. The output of the tool is a string that contains an SQL query in markdown format, the number of rows the query returns, and the first three rows.
pdf tool: This is a summarization and quality assurance tool that is used to answer questions about clinical trial protocols. For example, the tool can be used to identify which protocol documents the user wants to focus on and answers the users question. Questions about the protocol document can be related to get high level information about the trial objective, endpoint, eligibility, procedures, etc. Further, question can relate to a comparison between multiple protocols. The input to the tool is a query string. The output of the tool is a string the answer to the user question.
fda guidance tool: This is a summarization and quality assurance tool that is used to understand the guidance from FDA for a specific disease before writing a draft protocol or draft outline. The tool identified which FDA guidance documents are the most relevant and which sections are to have in mind before writing the protocol sections. Questions about the protocol document can be related to get information and guidance when writing objective, endpoint, eligibility, procedures, etc. The input to the tool is a query string. The output of the tool is a string the answer to the user question.
api tool: This tool is used as a search engine for clinical trials in ClinicalTrials.gov API. The input to this tool is a user question in natural language. The output of the tool is the selected fields of trials found by the search engine and total count.
drug labeling api tool: This tool is used as a search engine for drug labeling in FDA Drug Labeling API. The input to the tool is a user question in natural language. The output of the tool is the drug labeling information and total count.
drug adverse event api tool: This tool is used as a search engine for drug adverse event in FDA Drug Adverse Event API. The input of the tool is a user question in natural language. The output of the tool is the drug adverse event information and total count.

### Example Generative AI Modules

In general, the generative AI module 152 is a deep learning model that operates according to the principle of self-attention (e.g., a computer-specific technique that mimics cognitive attention). For example, the generative AI module 152 differentially weighs the significance of each part of an input (which includes the recursive output) data, and uses one or more attention mechanism to provide context for any position in the input sequence.

A generalized architecture of a generative AI module is described below.

### Input:

In general, input data strings are parsed into tokens (e.g., by a byte pair encoding tokenizer). Further, each token is converted via a word embedding into a vector. In some implementations, positional information of the token can be added to the word embedding.

### Encoder/Decoder Architecture:

In general, a generative AI module includes a decoder. Further, in some implementations, the generative AI module can also include an encoder. An encoder includes one or more encoding layers that process the input iteratively one layer after another, while the decoder includes one or more decoding layers that perform a similar operation with respect to the encoder's output.

Each encoder layer is configured to generate encodings that contain information about which parts of the inputs are relevant to each other, and passes these encodings to the next encoder layer as inputs. Each decoder layer performs the functional opposite, by taking all the encodings and using their incorporated contextual information to generate an output sequence. To achieve this, each encoder and decoder layer can make use of an attention mechanism.

For each part of the input, an attention mechanism weights the relevance of every other part and draws from them to produce the output. Each decoder layer has an additional attention mechanism that draws information from the outputs of previous decoders, before the decoder layer draws information from the encodings.

Further, the encoder and/or decoder layers can have a feed-forward neural network for additional processing of the outputs and contain residual connections and layer normalization steps.

As an example, one or more attention mechanism can be configured to implement scaled dot-product attention. For instance, when an input data string is passed into the generative AI module, attention weights can be calculated between every token simultaneously. An attention mechanism can produce embeddings for every token in context that contain information about the token itself along with a weighted combination of other relevant tokens each weighted by its attention weight.

For each attention unit, the generative AI module learns three weight matrices; the query weights W_{Q}, the key weights W_{K}, and the value weights W_{V}. For each token i, the input word embedding x₁ is multiplied with each of the three weight matrices to produce a query vector qᵢ=xᵢW_{Q}, a key vector kᵢ=xᵢW_{K}, and a value vector vᵢ=xᵢW_{V}. Attention weights are calculated using the query and key vectors: the attention weight aᵢⱼ from token i to token j is the dot product between qᵢ and kⱼ. The attention weights are divided by the square root of the dimension of the key vectors, \sqrt{dₖ}, which stabilizes gradients during training, and passed through a softmax which normalizes the weights. The fact that W_{Q} and W_{K} are different matrices allows attention to be non-symmetric: if token j (e.g., qᵢ·kⱼ is large), this does not necessarily mean that token j will attend to token i (e.g., qᵢ·kⱼ could be small). The output of the attention unit for token i is the weighted sum of the value vectors of all tokens, weighted by aᵢⱼ, the attention from token i to each token.

The attention calculation for all tokens can be expressed as one large matrix calculation using the softmax function, which is useful for training due to computational matrix operation optimizations that quickly compute matrix operations. The matrices Q, K, and V are defined as the matrices where the ith rows are vectors qᵢ, kᵢ, and vᵢ, respectively. Accordingly, attention can be presented as:

$$Attention ( Q , K , V ) = softmax ( \frac{{QK}^{T}}{\sqrt{{d}_{k}}} ) V$$

where softmax is taken over the horizontal axis.

In general, one set of (W_{Q}, W_{K}, W_{V}) matrices may be referred to as an attention head, and each layer in a generative AI module can have multiple attention heads. While each attention head attends to the tokens that are relevant to each token, with multiple attention heads the model can do this for different definitions of “relevance.”

In addition, the influence field representing relevance can become progressively dilated in successive layers. Further, the computations for each attention head can be performed in parallel, which allows for fast processing. The outputs for the attention layer are concatenated to pass into the feed-forward neural network layers.

### Encoder:

In general, encoder can include two major components: a self-attention mechanism and a feed-forward neural network. The self-attention mechanism accepts input encodings from the previous encoder and weights their relevance to each other to generate output encodings. The feed-forward neural network further processes each output encoding individually. These output encodings are then passed to the next encoder as its input, as well as to the decoders.

The first encoder takes positional information and embeddings of the input sequence as its input, rather than encodings.

The encoder is bidirectional. Attention can be placed on tokens before and after the current token.

A positional encoding is a fixed-size vector representation that encapsulates the relative positions of tokens within a target sequence.

The positional encoding is defined as a function of type f: [symbol] -> [symbol] ; d∈ [symbol] , d>0, where d is a positive even integer. The full position encoding can be represented as follows:

$$( {f ( t )}_{2 k} , {f ( t )}_{2 k} + 1 ) = ( sin ( θ ) , cos ( θ ) ) \qquad ∀ k ∈ \{ 0 , 1 , … , d / 2 - 1 \}$$

$$where$$

$$θ = \frac{t}{{r}_{k}} ,$$

$$r = {N}^{2 / d} .$$

Here, N is a free parameter that is significantly larger than the biggest k that would be input into the positional encoding function.

This positional encoding function allows the generative transformation model to perform shifts as linear transformations:

$$f ( t + Δ t ) = diag ( f Δ t ) ) f ( t )$$

where Δt∈ [symbol] is the distance one wishes to shift. This allows the transformer to take any encoded position, and find the encoding of the position n-steps-ahead or n-steps-behind, by a matrix multiplication.

By taking a linear sum, any convolution can also be implemented as linear transformations:

$${\sum }_{j} {c}_{j} f ( t + Δ {t}_{j} ) = ( {\sum }_{j} {c}_{j} diag ( f ( Δ {t}_{j} ) ) ) f ( t )$$

for any constants cⱼ. This allows the transformer to take any encoded position and find a linear sum of the encoded locations of its neighbors. This sum of encoded positions, when fed into the attention mechanism, would create attention weights on its neighbors, much like what happens in a convolutional neural network language model.

Although an example positional encoding technique is described above, in practice, other positional encoding techniques can also be performed, either instead or in addition to those described above. Further, in some implementations, the generative AI module need not perform positional encoding.

### Decoder:

Each decoder includes three major components: a self-attention mechanism, an attention mechanism over the encodings, and a feed-forward neural network. The decoder functions in a similar fashion to the encoder, but an additional attention mechanism is inserted which instead draws relevant information from the encodings generated by the encoders. This mechanism can also be called the encoder-decoder attention.

Like the first encoder, the first decoder takes positional information and embeddings of the output sequence as its input, rather than encodings. The transformer does not use the current or future output to predict an output, so the output sequence is partially masked to prevent this reverse information flow. This allows for autoregressive text generation. For all attention heads, attention cannot be placed on following tokens. The last decoder is followed by a final linear transformation and softmax layer, to produce the output probabilities.

Additional information regarding generative AI modules can be found in “Attention Is All You Need,” arXiv: 1706.03762 by Vaswani, et al., the contents of which are incorporated herein in their entirely.

### Example Processes

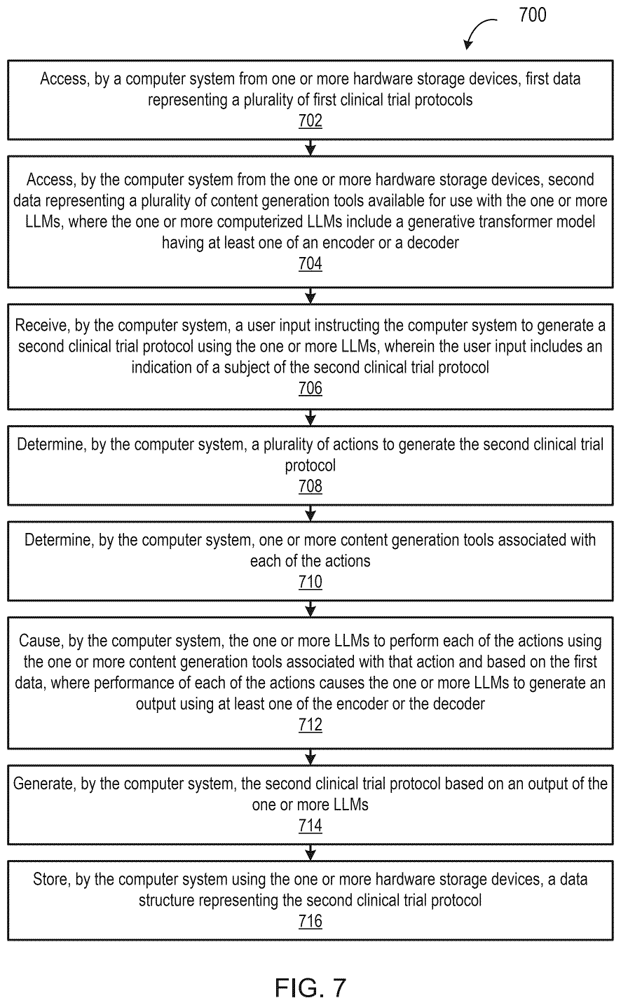

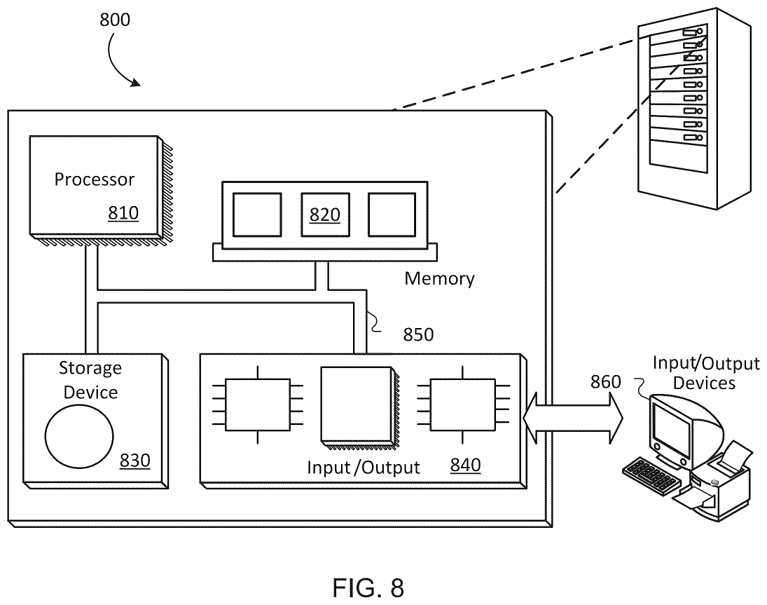

FIG. 7 shows an example process 700 for clinical trial documents using a generative AI system. In some implementations, the process 700 can be performed by the system 100 described in this disclosure (for example, the system 100 including the engine 150 shown and described with reference to FIGS. 1 and 2) using one or more processors (for example, using the processor or processors 810 shown in FIG. 8).

In the process 700, a system accessing, from one or more hardware storage devices, first data representing a plurality of first clinical trial protocols (702).

In some implementations, the first data can further represents a plurality of drugs and/or a plurality of medical treatments.

In some implementations, the first data can include structured data. Further, accessing the first data can include querying the structured data, normalizing the structured data, augmenting the structured data using the one or more LLMs, adding metadata to the structured data, and/or ingesting the structured data into a vector database.

In some implementations, the first data can include one or more documents. Further, accessing the first data can include parsing the one or more documents, augmenting the one or more documents, segmenting the one or more documents into one or more portions, adding metadata to the one or more documents using the one or more LLMs, and/or ingesting the documents into a vector database.

The system accesses, from the one or more hardware storage devices, second data representing a plurality of content generation tools available for use with the one or more LLMs (704). The one or more computerized LLMs include a generative transformer model having at least one of an encoder or a decoder.

The system receives a user input instructing the computer system to generate a second clinical trial protocol using the one or more LLMs (706). The user input includes an indication of a subject of the second clinical trial protocol.

In some implementations, the subject of the second clinical trial protocol can be one of a drug or a medical procedure.

The system determines a plurality of actions to generate the second clinical trial protocol (708).

In some implementations, the actions can include searching the first data for content relevant to second clinical trial protocol, generating a preview of the second clinical trial protocol, and/or generating the second clinical trial protocol.

The system determines one or more content generation tools associated with each of the actions (710).

In some implementations, the content generation tools can include a tool to perform a search of the first data, a tool to perform a search of one or more external databases, and/or a tool to perform a search of one or more websites.

In some implementations, the content generation tools can include a tool to generate a title of the second clinical trial protocol and/or a tool to generate one or more sections of the second clinical trial protocol.

In some implementations, the content generation tools can include a tool to generate a summary of the second clinical trial protocol.

In some implementations, the content generation tools can include a tool to generate at least one of a graph or a chart for the second clinical trial protocol.

In some implementations, the content generation tools can include a tool to prompt a user for additional user input regarding the second clinical trial protocol.

The system causes the one or more LLMs to perform each of the actions using the one or more content generation tools associated with that action and based on the first data (712). Performance of each of the actions causes the one or more LLMs to generate an output using at least one of the encoder or the decoder.

The system generates the second clinical trial protocol based on an output of the one or more LLMs (714).

The system stores, using the one or more hardware storage devices, a data structure representing the second clinical trial protocol (716).

In some implementations, the process 700 can further include presenting a graphical user interface representing the generation of the second clinical trial protocol. Presenting the graphical user interface can include continuously updating contents of the graphical user interface during the generation of the second clinical trial protocol. The graphical user interface can include a plurality of first graphical display elements, each of the first graphical display elements representing a different respective one of the actions. The graphical user interface can include a plurality of second graphical display elements, each of the second graphical display elements representing a summary of content generated for a different respective one of the actions.

In some implementations, the encoder and/or the decoder can be configured to apply a computerized attention mechanism over its respective inputs while generating the second clinical trial protocol.

In some implementations, the process 700 can further include (i) receiving a second user input instructing the computer system to generate one or more clinical trial documents using the one or more LLMs, (ii) determining a plurality of second actions to generate the one or more clinical trial documents, (iii) determining one or more second content generation tool associated with each of the second actions, and (iv) causing the one or more LLMs to perform each of the second actions using the one or more second content generation tools associated with that second action. Performance of each of the second actions can cause the one or more LLMs to generate a second output. Further, the process 700 can include (i) generating the one or more clinical trial documents based on the second output of the one or more LLMs, and (ii) storing, using the one or more hardware storage devices, a second data structure representing the one or more clinical trial documents.

In some implementations, the one or more clinical trial documents can include a clinical study report.

In some implementations, the one or more clinical trial documents can include an informed consent form.

### Example Computer Systems

FIG. 8 depicts an example computing system, according to implementations of the present disclosure. The system 800 may be used for any of the operations described with respect to the various implementations discussed herein. The system 800 may include one or more processors 810, a memory 820, one or more storage devices 830, and one or more input/output (I/O) devices 880 controllable through one or more I/O interfaces 840. The various components 810, 820, 830, 840, or 860 may be interconnected through at least one system bus 850, which may enable the transfer of data between the various modules and components of the system 800.

The processor(s) 810 may be configured to process instructions for execution within the system 800. The processor(s) 810 may include single-threaded processor(s), multi-threaded processor(s), or both. The processor(s) 810 may be configured to process instructions stored in the memory 820 or on the storage device(s) 830. The processor(s) 810 may include hardware-based processor(s) each including one or more cores. The processor(s) 810 may include general purpose processor(s), special purpose processor(s), or both.

The memory 820 may store information within the system 800. In some implementations, the memory 820 includes one or more computer-readable media. The memory 820 may include any number of volatile memory units, any number of non-volatile memory units, or both volatile and non-volatile memory units. The memory 820 may include read-only memory, random access memory, or both. In some examples, the memory 820 may be employed as active or physical memory by one or more executing software modules.

The storage device(s) 830 may be configured to provide (e.g., persistent) mass storage for the system 800. In some implementations, the storage device(s) 830 may include one or more computer-readable media. For example, the storage device(s) 830 may include a floppy disk device, a hard disk device, an optical disk device, or a tape device. The storage device(s) 830 may include read-only memory, random access memory, or both. The storage device(s) 830 may include one or more of an internal hard drive, an external hard drive, or a removable drive.

One or both of the memory 820 or the storage device(s) 830 may include one or more computer-readable storage media (CRSM). The CRSM may include one or more of an electronic storage medium, a magnetic storage medium, an optical storage medium, a magneto-optical storage medium, a quantum storage medium, a mechanical computer storage medium, and so forth. The CRSM may provide storage of computer-readable instructions describing data structures, processes, applications, programs, other modules, or other data for the operation of the system 800. In some implementations, the CRSM may include a data store that provides storage of computer-readable instructions or other information in a non-transitory format. The CRSM may be incorporated into the system 800 or may be external with respect to the system 800. The CRSM may include read-only memory, random access memory, or both. One or more CRSM suitable for tangibly embodying computer program instructions and data may include any type of non-volatile memory, including but not limited to: semiconductor memory devices, such as EPROM, EEPROM, and flash memory devices; magnetic disks such as internal hard disks and removable disks; magneto-optical disks; and CD-ROM and DVD-ROM disks. In some examples, the processor(s) 810 and the memory 820 may be supplemented by, or incorporated into, one or more application-specific integrated circuits (ASICs).

The system 800 may include one or more I/O devices 860. The I/O device(s) 860 may include one or more input devices such as a keyboard, a mouse, a pen, a game controller, a touch input device, an audio input device (e.g., a microphone), a gestural input device, a haptic input device, an image or video capture device (e.g., a camera), or other devices. In some examples, the I/O device(s) 860 may also include one or more output devices such as a display, LED(s), an audio output device (e.g., a speaker), a printer, a haptic output device, and so forth. The I/O device(s) 860 may be physically incorporated in one or more computing devices of the system 800, or may be external with respect to one or more computing devices of the system 800.

The system 800 may include one or more I/O interfaces 840 to enable components or modules of the system 800 to control, interface with, or otherwise communicate with the I/O device(s) 860. The I/O interface(s) 840 may enable information to be transferred in or out of the system 800, or between components of the system 800, through serial communication, parallel communication, or other types of communication. For example, the I/O interface(s) 840 may comply with a version of the RS-232 standard for serial ports, or with a version of the IEEE 1284 standard for parallel ports. As another example, the I/O interface(s) 840 may be configured to provide a connection over Universal Serial Bus (USB) or Ethernet. In some examples, the I/O interface(s) 840 may be configured to provide a serial connection that is compliant with a version of the IEEE 1394 standard.

The I/O interface(s) 840 may also include one or more network interfaces that enable communications between computing devices in the system 800, or between the system 800 and other network-connected computing systems. The network interface(s) may include one or more network interface controllers (NICs) or other types of transceiver devices configured to send and receive communications over one or more networks using any network protocol.

Computing devices of the system 800 may communicate with one another, or with other computing devices, using one or more networks. Such networks may include public networks such as the internet, private networks such as an institutional or personal intranet, or any combination of private and public networks. The networks may include any type of wired or wireless network, including but not limited to local area networks (LANs), wide area networks (WANs), wireless WANs (WWANs), wireless LANs (WLANs), mobile communications networks (e.g., 3G, 4G, Edge, etc.), and so forth. In some implementations, the communications between computing devices may be encrypted or otherwise secured. For example, communications may employ one or more public or private cryptographic keys, ciphers, digital certificates, or other credentials supported by a security protocol, such as any version of the Secure Sockets Layer (SSL) or the Transport Layer Security (TLS) protocol.

The system 800 may include any number of computing devices of any type. The computing device(s) may include, but are not limited to: a personal computer, a smartphone, a tablet computer, a wearable computer, an implanted computer, a mobile gaming device, an electronic book reader, an automotive computer, a desktop computer, a laptop computer, a notebook computer, a game console, a home entertainment device, a network computer, a server computer, a mainframe computer, a distributed computing device (e.g., a cloud computing device), a microcomputer, a system on a chip (SoC), a system in a package (SiP), and so forth. Although examples herein may describe computing device(s) as physical device(s), implementations are not so limited. In some examples, a computing device may include one or more of a virtual computing environment, a hypervisor, an emulation, or a virtual machine executing on one or more physical computing devices. In some examples, two or more computing devices may include a cluster, cloud, farm, or other grouping of multiple devices that coordinate operations to provide load balancing, failover support, parallel processing capabilities, shared storage resources, shared networking capabilities, or other aspects.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method for generating clinical trial protocols using one or more computerized large language models (LLMs), the method comprising:
accessing, by a computer system from one or more hardware storage devices, first data representing a plurality of first clinical trial protocols;
accessing, by the computer system from the one or more hardware storage devices, second data representing a plurality of content generation tools available for use with the one or more LLMs, wherein the one or more computerized LLMs comprise a generative transformer model having at least one of an encoder or a decoder;
receiving, by the computer system, a user input instructing the computer system to generate a second clinical trial protocol using the one or more LLMs, wherein the user input comprises an indication of a subject of the second clinical trial protocol;
determining, by the computer system, a plurality of actions to generate the second clinical trial protocol;
determining, by the computer system, one or more content generation tools associated with each of the actions;
causing, by the computer system, the one or more LLMs to perform each of the actions using the one or more content generation tools associated with that action and based on the first data, wherein performance of each of the actions causes the one or more LLMs to generate an output using at least one of the encoder or the decoder;
generating, by the computer system, the second clinical trial protocol based on an output of the one or more LLMs; and
storing, by the computer system using the one or more hardware storage devices, a data structure representing the second clinical trial protocol.

2. The method of claim 1, further comprising:
presenting a graphical user interface representing the generation of the second clinical trial protocol.

3. The method of claim 2, wherein presenting the graphical user interface comprises continuously updating contents of the graphical user interface during the generation of the second clinical trial protocol.

4. The method of claim 2, wherein the graphical user interface comprises a plurality of first graphical display elements, each of the first graphical display elements representing a different respective one of the actions.

5. The method of claim 2, wherein the graphical user interface comprises a plurality of second graphical display elements, each of the second graphical display elements representing a summary of content generated for a different respective one of the actions.

6. The method of claim 1, wherein the subject of the second clinical trial protocol is one of a drug or a medical procedure.

7. The method of claim 1, wherein the first data further represents at least one of:
a plurality of drugs, or
a plurality of medical treatments.

8. The method of claim 1, wherein the first data comprises structured data, and wherein accessing the first data comprises at least one of:
querying the structured data,
normalizing the structured data,
augmenting the structured data using the one or more LLMs,
adding metadata to the structured data, or
ingesting the structured data into a vector database.

9. The method of claim 1, wherein the first data comprises one or more documents, and
wherein accessing the first data comprises at least one of:
parsing the one or more documents,
augmenting the one or more documents,
segmenting the one or more documents into one or more portions,
adding metadata to the one or more documents using the one or more LLMs, or
ingesting the documents into a vector database.

10. The method of claim 1, wherein the actions comprise at least one of:
searching the first data for content relevant to second clinical trial protocol,
generating a preview of the second clinical trial protocol, or
generating the second clinical trial protocol.

11. The method of claim 1, wherein the content generation tools comprise at least one of:
a tool to perform a search of the first data,
a tool to perform a search of one or more external databases, or
a tool to perform a search of one or more websites.

12. The method of claim 1, wherein the content generation tools comprise at least one of:
a tool to generate a title of the second clinical trial protocol, or
a tool to generate one or more sections of the second clinical trial protocol.

13. The method of claim 1, wherein the content generation tools comprise:
a tool to generate a summary of the second clinical trial protocol.

14. The method of claim 1, wherein the content generation tools comprise:
a tool to generate at least one of a graph or a chart for the second clinical trial protocol.

15. The method of claim 1, wherein the content generation tools comprise:
a tool to prompt a user for additional user input regarding the second clinical trial protocol.

16. The method of claim 1, wherein at least one of the encoder or the decoder is configured to apply a computerized attention mechanism over its respective inputs while generating the second clinical trial protocol.

17. The method of claim 1, further comprising:
receiving a second user input instructing the computer system to generate one or more clinical trial documents using the one or more LLMs;
determining a plurality of second actions to generate the one or more clinical trial documents;
determining one or more second content generation tool associated with each of the second actions;
causing the one or more LLMs to perform each of the second actions using the one or more second content generation tools associated with that second action, wherein performance of each of the second actions causes the one or more LLMs to generate a second output;
generating the one or more clinical trial documents based on the second output of the one or more LLMs; and
storing, using the one or more hardware storage devices, a second data structure representing the one or more clinical trial documents.

18. The method of claim 17, wherein the one or more clinical trial documents comprise a clinical study report.

19. The method of claim 17, wherein the one or more clinical trial documents comprise an informed consent form.

20. A system, comprising:
at least one processor; and
a memory communicatively coupled to the at least one processor, the memory storing instructions which, when executed by the at least one processor, cause the at least one processor to perform operations comprising:
accessing, from one or more hardware storage devices, first data representing a plurality of first clinical trial protocols;
accessing, from the one or more hardware storage devices, second data representing a plurality of content generation tools available for use with the one or more LLMs, wherein the one or more computerized LLMs comprise a generative transformer model having at least one of an encoder or a decoder;
receiving a user input instructing the computer system to generate a second clinical trial protocol using the one or more LLMs, wherein the user input comprises an indication of a subject of the second clinical trial protocol;
determining a plurality of actions to generate the second clinical trial protocol;
determining one or more content generation tools associated with each of the actions;
causing the one or more LLMs to perform each of the actions using the one or more content generation tools associated with that action and based on the first data, wherein performance of each of the actions causes the one or more LLMs to generate an output using at least one of the encoder or the decoder;
generating the second clinical trial protocol based on an output of the one or more LLMs; and
storing, using the one or more hardware storage devices, a data structure representing the second clinical trial protocol.

21. One or more non-transitory computer-readable media storing instructions which, when executed by at least one processor, cause the at least one processor to perform operation comprising:
accessing, from one or more hardware storage devices, first data representing a plurality of first clinical trial protocols;
accessing, from the one or more hardware storage devices, second data representing a plurality of content generation tools available for use with the one or more LLMs, wherein the one or more computerized LLMs comprise a generative transformer model having at least one of an encoder or a decoder;
receiving a user input instructing the computer system to generate a second clinical trial protocol using the one or more LLMs, wherein the user input comprises an indication of a subject of the second clinical trial protocol;
determining a plurality of actions to generate the second clinical trial protocol;
determining one or more content generation tools associated with each of the actions;
causing the one or more LLMs to perform each of the actions using the one or more content generation tools associated with that action and based on the first data, wherein performance of each of the actions causes the one or more LLMs to generate an output using at least one of the encoder or the decoder;
generating the second clinical trial protocol based on an output of the one or more LLMs; and
storing, using the one or more hardware storage devices, a data structure representing the second clinical trial protocol.

22. A method for generating clinical study reports using one or more computerized large language models (LLMs), the method comprising:
accessing, by a computer system from one or more hardware storage devices, first data representing a plurality of first clinical study reports;
accessing, by the computer system from the one or more hardware storage devices, second data representing a plurality of content generation tools available for use with the one or more LLMs, wherein the one or more computerized LLMs comprise a generative transformer model having at least one of an encoder or a decoder;
receiving, by the computer system, a user input instructing the computer system to generate a second clinical study reports using the one or more LLMs, wherein the user input comprises an indication of a subject of the second clinical study reports;
determining, by the computer system, a plurality of actions to generate the second clinical study reports;
determining, by the computer system, one or more content generation tools associated with each of the actions;
causing, by the computer system, the one or more LLMs to perform each of the actions using the one or more content generation tools associated with that action and based on the first data, wherein performance of each of the actions causes the one or more LLMs to generate an output using at least one of the encoder or the decoder;
generating, by the computer system, the second clinical study reports based on an output of the one or more LLMs; and
storing, by the computer system using the one or more hardware storage devices, a data structure representing the second clinical study reports.
